(19) United States
(12) Patent Application Publication
(10) Pub. No.: US 2008/0280332 A1
(43) Pub. Date: Nov. 13, 2008

(54) Production of Resistant Starch Product Having Tailored Degree of Polymerization

(76) Inventor: Donald W. Harris, Springfield, IL (US)

Correspondence Address: WILLIAMS, MORGAN & AMERSON, P.C., 10333 RICHMOND, SUITE 1100, HOUSTON, TX, 77042, US

(21) Appl. No.: 12/038,986

(22) Filed: Feb. 28, 2008

Related U.S. Application Data
(60) Provisional application No. 60/894,825, filed on Mar. 14, 2007.
(60) Provisional application No. 60/893,160, filed on Mar. 6, 2007.

(51) Int. Cl. C12P 19/04

(52) U.S. Cl. 435/101

(57) ABSTRACT

A process for producing a starch comprises treating a feed starch that comprises amylopectin with glucanotransferase to produce a chain-extended starch, treating the chain-extended starch with a debranching enzyme to produce a starch product that comprises amylose fragments, crystallizing at least part of the starch product, heating the starch product in the presence of moisture, treating the starch product with alpha-amylase, and washing the starch product to remove at least some non-crystallized starch, wherein the degree of polymerization of the starch product is increased by increasing the amylose content of the feed starch or is decreased by decreasing the amylose content of the feed starch. The product of this process can have a relatively high total dietary fiber content, a relatively high heat resistance, or both.

This application claims priority from U.S. provisional patent application Ser. No. 60/894,825, filed on Mar. 14, 2007, and U.S. provisional patent application Ser. No. 60/893,160, filed on Mar. 6, 2007, both of which are incorporated herein by reference.

### BACKGROUND OF THE INVENTION

Starch comprises two polysaccharides: amylose and amylopectin. Amylose is a generally linear polymer that comprises glucose units connected by alpha 1-4 glycosidic linkages. Amylopectin is a branched polymer in which many of the glucose units are connected by alpha 1-4 glycosidic linkages, but some are connected by alpha 1-6 glycosidic linkages.

Alpha-amylase is an enzyme that is present in the human body and which hydrolyzes alpha 1-4 linkages in starch, thus leading to digestion of the starch. In certain situations it is desirable to produce starch that resists hydrolysis by alpha-amylase, for example to decrease the caloric content of the starch, or to increase its dietary fiber content. However, attempts to produce such starch in the past have suffered from one or more problems, such as high cost.

Amylase-resistant starch is usually produced from high-amylose starch, which is often expensive. There is a need for improved processes for producing starch with a high content of amylose that is suitable for production of alpha-amylase resistant starch.

### SUMMARY OF THE INVENTION

One embodiment of the invention is a process for producing a starch product that comprises (a) treating a feed starch with glucanotransferase to produce a chain-extended starch; (b) treating the chain-extended starch with a debranching enzyme to produce a starch product that comprises amylose fragments; (c) crystallizing at least part of the starch product; (d) heating the starch product in the presence of moisture; (e) treating the starch product with alpha-amylase; and (f) washing the starch product to remove at least some non-crystallized starch, wherein the degree of polymerization of the starch product is increased by increasing the amylose content of the feed starch or is decreased by decreasing the amylose content of the feed starch.

The process can also comprise recovering the remaining starch product after it has been washed. In some embodiments of the process, the feed starch is heated to at least partially gelatinize it prior to treatment with glucanotransferase.

In some embodiments of the process, at least about 38% by weight of the starch product comprises amylose fragments that have a degree of polymerization (DP) of at least about 35. The process can optionally further include recovering the amylose fragments. As another option, the process can include membrane filtering a solution or dispersion of the starch product to increase the concentration of amylose fragments that have a degree of polymerization (DP) of at least about 35.

Another embodiment of the present invention is a process for producing a starch product that comprises treating a feed starch with glucanotransferase to produce a chain-extended starch; treating the chain-extended starch with a debranching enzyme to produce a starch product that comprises amylose fragments; crystallizing at least part of the starch product; heating the starch product in the presence of moisture; and washing the starch product to remove at least some non-crystallized starch, wherein the degree of polymerization of the starch product is increased by increasing the amylose content of the feed starch or is decreased by decreasing the amylose content of the feed starch. Other than the absence of treatment with alpha-amylase, various embodiments of this process can be similar to or the same as those of the above-described process.

Another embodiment of the present invention is a starch product produced by any of the above-described processes. In some embodiments of the invention, at least about 40% by weight of the amylose fragments have a degree of polymerization (DP) of at least about 35. If the process used to make the starch product includes membrane filtration, then in some embodiments at least about 50% by weight of the amylose fragments have a degree of polymerization (DP) of at least about 35. In some instances the starch product has a peak melting temperature of greater than about 105° C.

Another embodiment of the invention is a food product that contains the above-described starch product.

### DESCRIPTION OF SPECIFIC EMBODIMENTS

One embodiment of the present invention is a process of producing starch having a relatively high content of amylose. This process includes treating a feed starch that comprises amylopectin with glucanotransferase to extend at least some of the starch chains, and treating the chain-extended starch with a debranching enzyme to produce amylose fragments. These amylose fragments can then be crystallized to produce a resistant starch product.

Ordinary dent corn starch can be debranched enzymatically to give short chain amylose fragments, but since the amylopectin component of the starch is usually composed of relatively short branched chains, the product contains too few of the longer chain lengths that are needed for enzyme resistance. Debranched dent corn starch that has not been modified with a glucanotransferase typically contains less than 35% of the DP35 and higher chain lengths (i.e., starch molecules having a degree of polymerization of at least 35) and therefore does not have the thermal stability needed for a resistant starch. In addition, the debranched dent starch contains a fraction of long chain lengths from amylose as well as short chains from amylopectin. This combination of heterogeneous chain lengths is not optimal for crystallization and amylase resistance.

The feed starch used in the present process can come from a variety of sources, including dent corn, waxy corn, high amylose ae genetic corn (ae is the name of a genetic mutation commonly known by corn breeders and is short for “amylose extender”), potato, tapioca, rice, pea, wheat, waxy wheat, as well as purified amylose from these starches, and alpha-1,4 glucans produced according to patent application WO 00/14249, which is incorporated herein by reference, and combinations of two or more of these starch sources. Chemically modified starches, such as hydroxypropyl starches, starch adipates, acetylated starches, and phosphorylated starches, can also be used in the present invention. For example, suitable chemically modified starches include, but are not limited to, crosslinked starches, acetylated and organically esterified starches, hydroxyethylated and hydroxypropylated starches, phosphorylated and inorganically esterified starches, cationic, anionic, nonionic, and zwitterionic starches, and succinate and substituted succinate derivatives of starch. Such modifications are known in the art, for example in Modified Starches: Properties and Uses, Ed. Wurzburg, CRC Press, Inc., Florida (1986). Other suitable modifications and methods are disclosed in U.S. Pat. Nos. 4,626,288, 2,613,206 and 2,661,349, which are incorporated herein by reference.

If the feed starch is a waxy starch, it can be at least partially debranched by treatment with a debranching enzyme prior to treatment with glucanotransferase. Suitable debranching enzymes for this purpose include pullulanase and isoamylase. This provides a source of fragments that will be transferred by the glucanotransferase to the amylopectin non-reducing ends, resulting in longer branched chains.

An improved resistant starch, with a more tailored degree of polymerization (DP) than that observed with use of dent corn starch alone, can be produced by modifying the feed starch, as will be described below.

For longer DP than that obtained by treating regular dent corn starch alone, amylose can be added, for example, amylose isolated from dent starch or high amylose starch. The DP correlates with the proportion of amylose in the feed starch, e.g., the longer the desired DP of the product, the greater the proportion of amylose that can be chosen for the feed starch. Using only a small amount of added amylose would result in only a small increase in DP compared to that obtained with dent corn starch alone.

Amylose can be isolated from dent starch by any appropriate technique. In one embodiment, amylose can be isolated from dent starch by a process comprising heating a slurry comprising dent starch, an organic solvent, and water to about 100° C. to about 200° C. under nitrogen; centrifuging the slurry, to yield an upper water phase and a lower amylose-organic solvent phase; heating a second slurry comprising the amylose-organic solvent phase and an alcohol-water mixture to about 100° C. to about 200° C., to yield an amylose-organic solvent-alcohol-water mixture; and drying the amylose-organic solvent-alcohol-water mixture under forced air at about 30° C. to about 70° C. for about 8 hr to about 24 hr, to yield amylose. In one embodiment, the organic solvent can be butanol and the alcohol-water mixture can be 3A alcohol (denatured ethanol) with 20% water.

Though not to be bound by theory, we submit a resistant starch product having a longer DP than that of dent corn starch would have more heat resistance as evidenced using differential scanning calorimetry (DSC) analysis where the peak temperature would be greater than about 115° C. and up to about 150° C. as more amylose is used in the starting mixture.

In addition, the heat/moisture treatment can be performed under conditions that give type B x-ray pattern for high resistance to α-amylase (high total dietary fiber (TDF)), while still having high DSC peak temperature and high resistance to heat treatment during possible food processing treatments.

For shorter DP starch, waxy corn starch can be used with added maltodextrin or corn syrup, such as 20 DE maltodextrin or 36 DE corn syrup. Though not to be bound by theory, we submit the final short chain starch amylose units produced by this method could be used for slowly digestible starch, which would be desirable in certain diets, such as for diabetics.

4-α-glucanotransferase [2.4.1.25] is an enzyme that catalyzes the transfer of a segment of a 1,4-alpha-D-glucan to a new position in an acceptor, which can be glucose or another 1,4-alpha-D-glucan. Glucanotransferase will catalyze the transfer of a maltosyl moiety to a maltotriose acceptor, releasing glucose. The glucose released can be used as a measurement of enzyme activity.

A suitable assay for determining glucanotransferase activity is as follows. In this assay, maltotriose is used as both substrate and acceptor molecule. Glucose is released in this reaction and can be measured after a modified version of the common glucose oxidase/peroxidase assay. (Werner, W. et al (1970) Z. Analyt. Chem. 252:224.) GOD-Perid solution can be obtained from a Glucose Release Kit from WAKO, or can be prepared with 65 mM sodium phosphate, pH 7 including 0.4 g/l glucose oxidase (Sigma G6125 or G7773), 0.013 g/l HRP (Sigma P8125), and 0.65 g/l ABTS (Calbiochem #194430). A 0.04 N NaOH solution is also used. The substrate solution is 1% maltotriose (0.1 g maltotriose in 10 ml of 50 mM phosphate buffer at pH 6.0).

### Standard Curve

Glucose solution: weight out 0.1806 g glucose into 500 ml MQ H₂O.

### Dilutions for Standard Curve

| Concentration | μL glucose solution | μL MQ water |
|---|---|---|
| 0.01 μmol | 5 | 495 |
| 0.05 μmol | 25 | 475 |
| 0.1 μmol | 50 | 450 |
| 0.25 μmol | 125 | 375 |
| 0.5 μmol | 250 | 250 |

120 μl of the substrate solution is pre-incubated at a selected temperature, e.g. 60° C., for 10 minutes. 20 μl of enzyme solution are added to the substrate solution and the reaction mixture is incubated at 60° for 10 minutes. The reaction is stopped by the addition of 20 μl of 0.04N NaOH. 20 μl is then transferred to a 96 well microtiter plate and 230 μl GOD-Perid solution is added. After 30 minutes at room temperature, the absorbance is measured at 420 nm. The enzyme activity is calculated relative to the standard curve of glucose in the range of 0-0.5 μmol glucose. One unit (U) of activity is defined as the amount of enzyme that liberates 1 μmol glucose/minute.

In some embodiments of the process, the glucanotransferase is used in a dosage of about 1-18,000 GTU per gram of feed starch. In other embodiments, the glucanotransferase is used in a dosage of about 10-18 GTU per gram of feed starch. Optionally, the glucanotransferase is used in a plurality of dosages that are supplied to the feed starch at separate times.

Treatment of the feed starch with glucanotransferase produces extensions of the chains on the amylopectin molecules. This treatment can be performed, for example, in aqueous solution or suspension at a temperature of about 70-100° C. and a pH of about 5.0 -8.5. As a result, the DP35 and higher content of the end product increases to over 38%, or in some cases to over 40%, and the chain lengths are much more uniform, which is indicated by a polydispersity of 2-4, compared to about 8 for debranched dent corn starch. In some embodiments of the invention, the dosage of glucanotransferase can be about 1-15 ml per 100 gram of starch, preferably about 5-12 ml/100 g. The glucanotransferase can be contacted with the starch in a single dose, or split into multiple doses. In one embodiment of the invention, the total dosage is split into three portions which are provided at separate times (for example, three separate doses of 2.5 ml/100 g each), with at least one hour between each. In some embodiments, the reaction temperature can be from about 75-85° C., and the reaction time can be less than about 8 hours, preferably less than about 6 hours.

Optionally, an additional starch-based material can be added to the chain-extended starch prior to debranching. For example, a maltodextrin can be added.

The resulting chain-extended starch can then be treated with a debranching enzyme, such as isoamylase or pullulanase, for example at a temperature of about 30-60° C. and a pH of about 4.0-5.0 to produce amylose fragments having desirable lengths. In some embodiments of the process, the debranching enzyme is used in a dosage of at least about 0.1 ml per gram of chain extended starch. In other embodiment, the debranching enzyme is used in a dosage of at least about at least about 1.0 ml per gram of chain extended starch. In certain embodiments of the invention, a dosage of isoamylase of about 1-10 mg per g of starch is used, preferably about 1-5 mg/g.

The DP35 and higher content can be enriched to over 50% by fractionation by microfiltration at an elevated temperature, such as about 60-120° C., more typically about 60-90° C., and even more typically 70-85° C. The debranched, glucanotransferase-treated, starch product after microfiltration can have a peak melting temperature greater than about 105° C., and can contain at least about 80% by weight resistant starch after heating in water to about 98° C.

Optionally, the debranched starch produced in step (b) is gelatinized in a jet cooker to solubilize the starch, and then is cooled to about 20-90° C. to crystallize.

Optionally, the product starch can be heat treated in the presence of moisture at a temperature of at least about 90° C., or in some embodiments at least about 98° C. In some embodiments of the process, in step (d) the starch product is heated to about 100-150° C. at a moisture content of about 15-35% by weight. In other embodiments, in step (d) the starch product is heated to about 120-130° C. at a moisture content of about 22-26% by weight. This heat-moisture treatment can increase the total dietary fiber (TDF) content and/or the resistant starch (RS) content of the starch product in some instances. For example, in some embodiments, the starch product has a total dietary fiber (TDF) content of at least about 10% by weight before the heat moisture treatment in step (d), or, in some instances, a TDF content greater than about 30% by weight before the heat moisture treatment in step (d). In some embodiments, the starch product has a TDF content of at least about 50% by weight after the heat moisture treatment of step (d), or, in some cases, a TDF content of greater than about 75% by weight after the heat moisture treatment of step (d). In some embodiments, the starch product has a resistant starch (RS) content of at least 40% by weight before the heat moisture treatment of step (d), and, in some cases, a RS content greater than about 80% by weight after the heat moisture treatment of step (d).

The heat moisture treatment can increase the TDF (AOAC 991.43) of the starch from about 15-35% to about 75-80% in some embodiments of the invention.

In one embodiment of the process, the feed starch is slurried in water at 15% solids and the pH is adjusted to 5.5 with dilute NaOH. The slurry is placed in an autoclave and heated to 140° C. for 30 minutes. After cooling to 85° C. and adjusting the pH to 5.5, glucanotransferase is added and allowed to react for 24 hours. The enzyme is deactivated by reducing the pH to below 3.0. The starch is redispersed by heating to 140° C. for one hour and then cooled to 45° C., and the pH is adjusted to 4.5. Isoamylase is added and allowed to react for 18-24 hours. The mixture is heated to 85° C. for one hour to deactivate the enzyme. If necessary, the product can be treated again with isoamylase by repeating the 140° C. heating and enzyme treatment at 45° C. and pH 4.5. The product can then be fractionated to increase the content of longer chain components. This can be carried out, for example, by microfiltration or ultrafiltration of the crystallized debranched product at a temperature of at least about 80° C. using a ceramic membrane with a pore size of about 0.45 microns. After collecting 1.5 to 2.5 volumes of permeate relative to the volume of the starting slurry, while maintaining the volume of the retentate by addition of deionized water, the product is isolated by concentrating and spray drying or by centrifuging and oven drying the retentate.

In another embodiment of the process, a starch product that comprises a substantial percentage of resistant starch can be produced by (a) treating a feed starch with glucanotransferase to produce a chain-extended starch; (b) treating the chain-extended starch with a debranching enzyme to produce a starch product that comprises amylose fragments; (c) crystallizing at least part of the starch product; (d) heating the starch product in the presence of moisture; (e) treating the starch product with alpha-amylase; and (f) washing the starch product. The remaining starch product can be recovered after it has been washed (i.e., after at least some of the non-crystallized components, and preferably the majority of such components, are removed by the washing). In many cases, the feed starch is heated to at least partially gelatinize it prior to treatment with glucanotransferase.

The heat/moisture treatment in step (d) helps to increase the percentage of total dietary fiber (TDF) and resistant starch (RS) in the starch product. Resistant starch content was analyzed using the method of Englyst et al. (Eur. J. Clinical Nut. (1992) 46 (Suppl. 2), S33-S50, “Classification and Measurement of Nutritionally Important Starch Fractions”). (All references in this patent to a percentage of resistant starch in a material are as determined by the Englyst assay.)

As an example of suitable conditions for this step, the starch product can be heated to about 120-150° C. with a beginning moisture content of about 20-35% by weight, for a time of about 1-12 hours. In some embodiments of the invention, the starch product is heated to about 125-135° C. with a beginning moisture content of about 25-27% by weight. At the conclusion of this step, in some embodiments of the process, the starch product will have a TDF content of about 70-80% by weight, a DSC enthalpy of about 22 Joules/gram, and good thermal stability.

The additional steps of treating the starch product with alpha-amylase and washing can increase the TDF content by removing at least some non-crystallized starch. The non-crystallized material tends to be more susceptible to degradation by amylase, and therefore its removal will usually boost the TDF and RS values of the product. In some embodiments, at the conclusion of these additional steps, at least about 50% by weight of the recovered starch product is oligomers having a degree of polymerization (DP) from about 24-100 (inclusive), and in some cases, at least about 75% by weight of the recovered starch product has a DP from about 24-100. In some embodiments, the recovered starch product has an enthalpy as measured by differential scanning calorimetry of at least about 20 Joules/gram. In some embodiments, the recovered starch product has a peak melting temperature of greater than about 105° C., a TDF content of at least about 85% by weight, and an enthalpy as measured by differential scanning calorimeter of at least about 27 Joules/gram. In certain embodiments, the starch product has a TDF value of 85-90% by weight and a DSC enthalpy of about 28 Joules/gram.

One advantage of the process is that it can produce a high TDF starch product from dent corn, and does not require a feed starch with unusually high amylose content. This makes the process more economical.

The product produced by the process contains a high percentage of amylose that is resistant to alpha-amylase. The resistant starch can be added to a number of food products to reduce their caloric density and glycemic index, and increase dietary fiber and probiotic effect in the colon.

Starch produced by this process can be used as a bulking agent or flour substitute in foods, such as reduced calorie baked goods. The starch is also useful for dietary fiber fortification in foods. Specific examples of foods in which the starch can be used include bread, cakes, cookies, crackers, extruded snacks, soups, frozen desserts, fried foods, pasta products, potato products, rice products, corn products, wheat products, dairy products, nutritional bars, food for diabetics, and beverages.

The starch product, at least in some embodiments, is thermally stable in water at a temperature of at least about 90° C., or in some cases at least about 100° C., allowing it to be used in food products that will be processed at high temperature and moisture conditions.

In some embodiments, the starch product has a crystal morphology (as determined by wide angle X-ray diffraction techniques) of A form, B form, or a combination thereof. In other words, the product can comprise 100% A form crystals, 100% B form crystals, or any blend of the two forms.

Certain embodiments of the invention are described in the following example.

### Example 1

### Preparation of Heat/Moisture Treated Resistant Starch With Tailored DP

The following example illustrates reactions designed to produce resistant starch with higher DP and heat stability.

Into a vessel would be added 125 lb of regular dent corn starch, 125 lb of amylose isolated from dent corn starch and 1420 lb water to give 15% starch slurry.

The starch slurry would be jet cooked at approximately 150 to 160° C. at a feed rate of approximately 2.0 gpm and the resulting paste flashed into a tank and maintained at approximately 88° C., with agitation.

Into the resulting starch paste as it entered the tank, would be injected a total of approximately 8,000 GTU/lb starch of 4-α-glucanotransferase enzyme (obtained from Novozymes) spread over the entire time period the paste would be pumped into the tank. The mixture would be allowed to react 3 hr at 88° C. with agitation.

Dilute sulfuric acid would be added to adjust the pH to 3.8-3.9 and the reactor contents cooled rapidly to approximately 55° C. by pumping through a heat exchanger into an agitated tank maintained at 55° C. To the slurry would be added 0.1 ml/100 g of starch of isoamylase enzyme obtained from Hayashibara and the enzyme would be allowed to react 16 hr at 55° C. while maintaining the pH at 3.8-3.9

The slurry would then be jet cooked at approximately 150° C. and allowed to cool slowly with stirring to 55° C. then held at 55° C. overnight to promote crystal formation.

The slurry would then be dewatered on a basket centrifuge and dried overnight in a tray dryer to approximately 10% moisture content. The resistant starch product would be ground to pass through a US #40 mesh sieve and labeled.

To 55 lb of resistant starch from the step above, with agitation, would be added sufficient water to give 25% total water content. The starch cake would be placed in a steam jacketed Littleford Reactor and heated with agitation in a nitrogen atmosphere at approximately 126° C. for 2 hr. The mixture would then be cooled and taken from the Littleford Reactor and tray dried to approximately 10% moisture content. The resulting heat/moisture treated resistant starch product would be ground to pass through a US #40 mesh sieve and labeled.

For somewhat smaller final DP, we might start with a mixture of 75% dent starch and 25% amylose or high amylose starch. For even longer DP final product, we might start with 30% dent corn starch and 75% amylose or high amylose corn starch. We could tailor the DP depending on the selected proportion of dent corn starch and amylose or high amylose corn starch used in the starting mixture.

### Example 2

### Preparation of Low-DP Starch

The following example illustrates reactions designed to produce starch with lower average degree of polymerization (DP) than would be obtained for waxy corn starch alone.

Into a vessel would be added 125 lb of waxy corn starch, 125 lb of 20 DE maltodextrin or 36 DE corn syrup and 1420 lb water to give 15% starch slurry.

The starch slurry would be jet cooked at approximately 150 to 160° C. at a feed rate of approximately 2.0 gpm and the resulting paste flashed into a tank and maintained at approximately 88° C., with agitation.

Into the resulting starch paste as it entered the tank, would be injected a total of approximately 8,000 GTU/lb starch of 4-α-glucanotransferase enzyme (obtained from Novozymes) spread over the entire time period the paste would be pumped into the tank. The mixture would be allowed to react 3 hr at 88° C. with agitation.

Dilute sulfuric acid would be added to adjust the pH to 3.8-3.9 and the reactor contents cooled rapidly to approximately 55° C. by pumping through a heat exchanger into an agitated tank maintained at 55° C. To the slurry would be added 0.1 ml/100 g of starch of isoamylase enzyme obtained from Hayashibara and the enzyme would be allowed to react 16 hr at 55° C. while maintaining the pH at 3.8-3.9

The slurry would then be jet cooked at approximately 150° C. and allowed to cool slowly with stirring to 55° C. then held at 55° C. overnight to promote crystal formation.

The slurry would then be dewatered on a basket centrifuge and dried overnight in a tray dryer to approximately 10% moisture content. The resistant starch product would be ground to pass through a US #40 mesh sieve and labeled.

To 55 lb of resistant starch from the step above, with agitation, would be added sufficient water to give 25% total water content. The starch cake would be placed in a steam jacketed Littleford Reactor and heated with agitation in a nitrogen atmosphere at approximately 126° C. for 2 hr. The mixture would then be cooled and taken from the Littleford Reactor and tray dried to approximately 10% moisture content. The resulting heat/moisture treated resistant starch product would be ground to pass through a US #40 mesh sieve and labeled.

For higher final DP we might start with a mixture of 75% waxy starch and 25% of 20 DE maltodextrin or 36 DE corn syrup. We could tailor make the DP depending on the selected proportion of waxy corn starch and 20 DE maltodextrin or 36 DE corn syrup used in the starting mixture.

### Example 3

### Procedure for Isolation of Amylose From Regular Dent Corn Starch

Into a 4-liter beaker was placed 176 g dry basis of regular dent corn starch and 176 g butanol. Water was added to give a total of 2200 g of total slurry. The slurry was placed in a 2-liter stainless steel pressure reactor and the air was removed with nitrogen gas (three purges at 55 psig nitrogen gas). The reactor contents were heated to 150° C. and held there for 15 min then cooled to room temperature. The slurry was stirred overnight at room temperature then centrifuged in 250 ml centrifuge tubes @˜3500 rpm in a table-top IEC centrifuge. The liquid was decanted leaving the sediment of amylose-butanol inclusion complex. Water was added and the sediment again centrifuged and the liquid decanted. To the sediment was added 1625 g of 80% denatured ethanol with 20% water and the slurry was heated to 150° C. for 5 min. in the stainless steel pressure reactor then cooled to room temperature. The slurry was filtered on a Buchner funnel then once again processed in 1625 g of additional 80% alcohol at 150° C. as before. The resulting amylose was dried in a forced air oven at 50° C. overnight yielding 21% of the starting dent corn starch dry weight. Analysis showed less than 100 ppm residual butanol. Amylose value by blue value was between 90-100%.

The preceding description of specific embodiments of the invention is not intended to be a list of every possible embodiment of the invention. Persons skilled in the art will recognize that other embodiments would be within the scope of the following claims.

What is claimed is:

1. A process for producing a starch product, comprising (a) treating a feed starch with glucanotransferase to produce a chain-extended starch; (b) treating the chain-extended starch with a debranching enzyme to produce a starch product that comprises amylose fragments; (c) crystallizing at least part of the starch product; (d) heating the starch product in the presence of moisture; (e) treating the starch product with alpha-amylase; and (f) washing the starch product to remove at least some non-crystallized starch,
wherein the degree of polymerization of the starch product is increased by increasing the amylose content of the feed starch or is decreased by decreasing the amylose content of the feed starch.

2. The process of claim 1, wherein the degree of polymerization of the starch product is increased and the differential scanning calorimetry (DSC) peak temperature of the starch product is from about 115° C. to about 150° C.

3. The process of claim 1, wherein the degree of polymerization of the starch product is increased and step (d) is performed under conditions that increase the resistance of the starch product to α-amylase.

4. The process of claim 1, wherein the feed starch contains a high-amylose material selected from the group consisting of amylose isolated from dent starch, high amylose starch, and mixtures thereof.

5. The process of claim 4, wherein the amylose isolated from dent starch is isolated by a process comprising:
heating a slurry comprising dent starch, an organic solvent, and water to about 100° C. to about 200° C. under nitrogen;
centrifuging the slurry, to yield an upper water phase and a lower amylose-organic solvent phase;
heating a second slurry comprising the amylose-organic solvent phase and an alcohol-water mixture to about 100° C. to about 200° C., to yield an amylose-organic solvent-alcohol-water mixture; and
drying the amylose-organic solvent-alcohol-water mixture under forced air at about 30° C. to about 70° C. for about 8 hr to about 24 hr, to yield amylose.

6. The process of claim 1, wherein the feed starch contains a low-amylose material selected from the group consisting of maltodextrin, corn syrup, and mixtures thereof.

7. A process for producing a starch product, comprising (a) treating a feed starch with glucanotransferase to produce a chain-extended starch; (b) treating the chain-extended starch with a debranching enzyme to produce a starch product that comprises amylose fragments; (c) crystallizing at least part of the starch product; (d) heating the starch product in the presence of moisture; and (e) washing the starch product to remove at least some non-crystallized starch,
wherein the degree of polymerization of the starch product is increased by increasing the amylose content of the feed starch or is decreased by decreasing the amylose content of the feed starch.

8. The process of claim 7, wherein the degree of polymerization of the starch product is increased and the differential scanning calorimetry (DSC) peak temperature of the starch product is from about 115° C. to about 150° C.

9. The process of claim 7, wherein the degree of polymerization of the starch product is increased and step (d) is performed under conditions that increase the resistance of the starch product to α-amylase.

10. The process of claim 7, wherein the feed starch contains a high-amylose material selected from the group consisting of amylose isolated from dent starch, high amylose starch, and mixtures thereof.

11. The process of claim 10, wherein the amylose isolated from dent starch is isolated by a process comprising:
heating a slurry comprising dent starch, an organic solvent, and water to about 100° C. to about 200° C. under nitrogen;
centrifuging the slurry, to yield an upper water phase and a lower amylose-organic solvent phase;
heating a second slurry comprising the amylose-organic solvent phase and an alcohol-water mixture to about 100° C. to about 200° C., to yield an amylose-organic solvent-alcohol-water mixture; and
drying the amylose-organic solvent-alcohol-water mixture under forced air at about 30° C. to about 70° C. for about 8 hr to about 24 hr, to yield amylose.

12. The process of claim 7, wherein the feed starch contains a low-amylose material selected from the group consisting of maltodextrin, corn syrup, and mixtures thereof.
